(19) United States
(12) Patent Application Publication
(10) Pub. No.: US 2008/0241264 A1
(43) Pub. Date: Oct. 2, 2008

(54) System and methods for collective nanorobotics for medical applications

(75) Inventor: Neal Solomon, Oakland, CA (US)

Correspondence Address: Neal Solomon, P.O. Box 21297, Oakland, CA, 94620, US

(73) Assignee: Solomon Research LLC, Oakland, CA (US)

(21) Appl. No.: 11/985,046

(22) Filed: Nov. 13, 2007

Related U.S. Application Data
(60) Provisional application No. 60/865,605, filed on Nov. 13, 2006.
(60) Provisional application No. 60/912,133, filed on Apr. 16, 2007.
(60) Provisional application No. 60/941,600, filed on Jun. 1, 2007.
(60) Provisional application No. 60/958,466, filed on Jul. 7, 2007.

(51) Int. Cl. A61K 9/51; G06F 19/00

(52) U.S. Cl. 424/490; 700/245; 424/422

(57) ABSTRACT

The invention discloses the use of collectives of nanorobots (CNRs) for medical applications. CNRs are used (a) to map the human body, (b) to regulate the cardio-vascular system, (c) for insulin regulation, (d) for targeted drug delivery, (e) for diagnosis of cellular pathologies and (f) for destroying tumor cells.

### CROSS-REFERENCES TO RELATED APPLICATIONS

The present application claims the benefit of priority under 35 U.S.C. § 119 from U.S. Provisional Patent Application Ser. No. 60/865,605, filed on Nov. 13, 2006, U.S. Provisional Patent Application Ser. No. 60/912,133, filed Apr. 16, 2007, U.S. Provisional Patent Application Ser. No. 60/941,600, filed Jun. 1, 2007 and U.S. Provisional Patent Application No. 60/958,466, filed Jul. 7, 2007, the disclosures of which are hereby incorporated by reference in their entirety for all purposes.

### FIELD OF THE INVENTION

The present invention pertains to the field of nanotechnology and nanorobotics. The system deals with epigenetic robotics applied to collectives of nanorobots. Specifically, the invention relates to nanoelectromechanical systems (NEMS) and microelectromechanical systems (MEMS), nanomechatronics and bionanomechatronics. The invention also deals with the coordination of collectives of nanorobots, synthetic nanorobotics and synthetic bionanorobotics, including synthetic assemblies of NEMS and nanorobots and synthetic nano-scale and micron-scale machine assembly processes. Applications of these systems and processes are made to bionanotechnology and nanomedicine.

### BACKGROUND OF THE INVENTION

To date, four waves, or generations, of nanotechnology have evolved. The first generation was comprised mainly of developments involving chemical composition, such as new nanomaterials. The second generation developed simple tubes and filaments by positioning atoms from the ground up with novel machinery. The third generation developed nanodevices that perform specific functions, such as nanoparticles for the delivery of chemicals. Finally, the fourth wave has developed self-assembling nanoentities by chemical means.

The present invention represents a fifth generation of self-organizing collectives of intelligent nanorobotics. Self-organizing processes are possible at the nano- and micron-level because of the convergence of nanoelectronics developments and nanomechatronics developments.

While the first four generations of nanotechnology have been developed by theoretical scientists and inventors, the fifth generation of nanotechnology has been largely open until now. The present invention fills the gaps in the literature and in the prior art involving nanorobotics.

Early twentieth century theoretical physicists discovered that the simplest atoms were measurable at the nanometer scale of one billionth of a meter. In 1959, in his lecture “Race to the Bottom,” the physicist Richard Feynman proposed a new science and technology to manipulate molecules at the nanoscale. In the 1970s Drexler's pioneering research into nanotechnology molecular-scale machinery provides a foundation for current research. In 1979, researchers at IBM developed scanning tunneling microscopy (STM) with which they manipulated atoms to spell the letters IBM. Also in the 1970s Ratner and his team at Northwestern developed the first nano-scale transistor-like device for nanoelectronics, which was developed into nanotransistors by researchers at the University of California at Berkeley in 1997. Researchers at Rice, Yale and Penn State were able to connect blocks of nanodevices and nanowires, while researchers at Hewlett Packard and UCLA were able to develop a computer memory system based on nano-assembly. Additionally, government researchers at NASA, NIST, DARPA and Naval Research have ongoing nanotechnology development projects, though these are mainly focused on nanoelectronics challenges. Finally, researchers at MIT, Cal Tech, USC, SUNY, Cornell, Maryland, Ill. and other universities in the U.S. have been joined by overseas researchers in developing novel nanotechnologies in order to meet Feynman's challenge.

Nanotech start-up ventures have sprung up to develop nanoscale crystals, to use as biological labels, for use in tagging proteins and nucleic acids (Quantum Dot) and to develop micro-scale arms and grippers by using MEMS to assemble manufacturing devices (Zyvex). Additionally, Nanosys, Nanometrics, Ultatech, Molecular Electronics, Applied Nanotech and Nanorex are ventures that have emerged to develop products in the nanotechnology market space. Until now, however, most of these businesses have focused on inorganic nanomaterials. Though a new generation of materials science has been aided by these earlier generations of nanotechnologies, the real breakthrough lies in identifying methods of developing intelligent systems at the nano-scale.

The two main models for building nanotechnology applications are the ground up method of building entities, on the one hand, and the bottom down method of shrinking photolithography techniques to the nanoscale. Both models present challenges for scientists.

In the case of the bottom up models, several specialized tools have been required. These include (a) atomic force microscopy (AFM), which uses electronics to measure the force exerted on a probe tip as it moves along a surface, (b) scanning tunneling microscopy (STM), which measures electrical current flowing between a scanning tip and a surface, (c) magnetic force microscopy (MFM), which uses a magnetic tip that scans a surface and (d) nanoscale synthesis (NSL), which constructs nanospheres.

In the case of the top down models, several methods and techniques have been developed, including (a) x-ray lithography, (b) ion beam lithography, (c) dip pen nanolithography (DPN), in which a “reservoir of ‘ink’ (atoms/molecules) is stored on top of the scanning probe tip, which is manipulated across the surface, leaving lines and patterns behind” (Ratner, 2003) and (d) micro-imprint lithography (MIL), which emulates a rubber stamp. Lithography techniques generally require the creation of a mask of a main model, which is then reproduced onto a substrate much like a semiconductor is manufactured. It is primarily through lithographic techniques that mass quantities of nanoentities can be created efficiently and cost-effectively.

The main patents obtained in the U.S. in the field of nanotechnology have focused on nanomaterials, MEMS, micro-pumps, micro-sensors, micro-voltaics, lithography, genetic microarray analysis and nano-drug delivery. Examples of these include a meso-microelectromechanical system package (U.S. Pat. No. 6,859,119), micro-opto-electro-mechanical systems (MOEMS) (U.S. Pat. No. 6,580,858), ion beam lithography system (U.S. Pat. No. 6,924,493), carbon nanotube sensors (U.S. Pat. No. 7,013,708) and microfabricated elastomeric valve and pump systems (U.S. Pat. Nos. 6,899,137 and 6,929,030). Finally, patents for a drug targeting system (U.S. Pat. No. 7,025,991) and for a design of artificial genes for use as controls in gene expression analytical system (U.S. Pat. No. 6,943,242), used for a DNA microarray, are applied to biotechnology. For the most part, these patents represent third and fourth generation nanotechnologies.

A new generation of nanotechnologies presents procedures for objects to interact with their environment and solve critical problems on the nano- and micron-scale. This generation of technology involves social intelligence and self-organization capabilities.

Biological analogies help to explain the performance of intelligent or self-organizing nanoentities. In the macro-scale environment, the behaviors of insects provides an important model for understanding how to develop models that emulate social intelligence in which chemical markers (pheromones) are used by individual entities to communicate a social goal. On the micro-scale, microbes and pathogens interoperate with the animal's immune system, in which battles either won or, lost determine survival of the host. Other intracellular models show how proteins interact in order to perform a host of functions. At the level of DNA, RNA transcription processes are highly organized methods for developing cellular reproduction. These micromachinery processes and functions occur at the nanoscale and provide useful analogies for nanotechnologies.

In order to draw on these biological system analogies, complexity theory has been developed in recent years. Researchers associated with the Sante Fe Institute have developed a range of theoretical models to merge complexity theory and biologically-inspired processes, including genetic algorithms and collective behavior of economic agents.

Such a new nanotechnology requires distributed computation and communication techniques. It is, moreover, necessary for such a technology to adapt to feedback from its environment. The present invention presents a system in which these operations occur and specifies a range of important applications for electronics, medicine and numerous other areas. The main challenges to this advanced nanotechnology system lie in the discovery of solutions to the problems of limited information, computation, memory, communication, mobility and power.

Challenges

The development of a fifth generation of nanotechnologies faces several challenges. First, the manufacturing of nanoparts is difficult. Second, the assembly of nanoparts into functional devices is a major challenge. Third, the grouping and coordination of collectives of nanodevices is problematic. Fourth, the control an d management of nanosystems is complex. Fifth, controlling the interaction of nanorobots in a collective system with its environment is formidable. Since physical properties operate differently at the nano-scale than at the macro-scale, we need to design systems that accommodate these unique physical forces.

The dozens of problems to identify include how to:

Build nanorobots

Connect nanodevices

Develop a nanorobotic power source

Develop nanorobotic computation

Develop specific nanorobotic functionality

Develop nanorobotic communication system(s)

Develop multi-functional nanorobotics

Develop systems in which nanorobots work together

Identify distinctive nanorobotic collective behaviors for specific applications

Activate nanorobotic functionality

Develop nanorobotic computer programming

Develop an external tracking procedure for a nanorobot

Develop an external activation of a nanorobot

Develop a hybrid control system for nanorobots

Use AI for nanorobots

Organize the behavior of nanorobot teams

Reorganize nanorobotic aggregates as teams adapt to environmental feedback

Obtain environmental inputs via sensors

Organize competing teams of nanorobots

Organize cooperating teams of nanorobots

Organize nanorobotic teams to anticipate behaviors

Organize nanorobotic teams to emulate biological processes such as the immune system

Developing Solutions to these Problems

Most prior technological innovations for nano-scale problems have focused on the first generations of nanotechnology and on materials science. The next generation focuses on intelligent systems applied to the nano entities. This fifth generation of innovation combines the development of nano-scale entities with intelligence and the collective behaviors of complex systems.

Few researchers have devised solutions to these complex nano-scale problems. Cavalcanti has developed theoretical notions to develop a model of collective nanorobotics. However, these solutions are not practical and will not work in real situations. For example, there is not enough power of mobility in this model to overcome natural forces. Similarly, according to this theoretical approach, autonomous computation resources of nanorobots are insufficient to perform even the simplest functions, such as targeting. Without computation capacity, AI will not work at this level; without AI there is no possible way to perform real-time environmental reaction and interaction.

Cavalcanti's 2D and 3D simulations are dependent on only several variable assumptions and will not withstand the “chaos” of real environmental interactive processes. In addition, the structure of these nanorobots cannot be built efficiently from the bottom up and still retain critical functionality. Even if these many problems can be solved, individual nanorobots cannot be trusted to behave without error inside cells. In other words, this conceptual generation of medical nanorobots may do more harm than good, particularly if they are not controllable.

The emerging field of epigenetic robotics deals with the relations between a robot and its environment. This field suggests that it is useful to program a robot to learn autonomously by interacting with its environment. However, these models do not apply to collective robotics in which it is necessary to learn from and interact with many more variables in the robots' environment, including other robots. In the case of collectives of nanorobots with resource constraints, the present invention adds volumes to this promising field.

Solomon's research in developing hybrid control systems for collective robotics systems and in developing novel approaches for molecular modeling systems presents pathways to solving these complex problems. These novel research streams are used in the present invention.

Prior systems of collective robotics generally do not address the complexities of nanotechnology. The behavior-based robot system using subsumption methods developed by Brooks at MIT is useful for managing individual robot behavior with limited computation capacity. On the other end of the spectrum, central control robotic systems require substantial computation resources. Hybrid control robotic systems synthesize elements from these two main control processes. Even more advanced robotic control systems involve the integration of a multi-agent software system with a robotic system that is particularly useful in controlling collectives of robots. This advanced collective robotic control system experiences both the benefits and detriments of the behavior-based model and the central control model.

Recent developments in collective robotics have borrowed inspiration from complex biological processes. Complex social behaviors such as flocking, herding and schooling have been studied, with ant algorithms representing the state of the art in computationally emulating and optimizing natural processes. Even more complex natural behaviors at the molecular level are discovered as we learn more about protein interactions. Specifically, the human immune system is a fascinating dynamic interactive network that has evolved over many years. Our challenge is to develop artificial mechanisms to surpass not only ant algorithms, which use the collective behavior of autonomous individuals that use chemical communications methods, but also the interactive workings of the human immunological system.

One of the main methods to develop these complex artificial network models for use in robotic systems is to use evolutionary computation, which emulates biological processes of evolution. Methods such as genetic algorithms or genetic programs emulate the behavior of generations of populations in order to solve complex problems. Similarly, artificial neural network approaches emulate the ability of the human brain to adapt to its environment in order to solve complex problems.

The development of cooperating collectives of robots in a network borrows inspiration from these biological systems. A team of interacting agents takes inspiration from the effective operation of a beehive or an ant colony in which specialist roles and coordination of tasks occur among thousands of agents. These complex network systems use self-organizing models of behavior to aggregate (combine into groups), to reaggregate and to adapt to their environment. However, there are limits to these models because of the constraints of communication, coordination, “computation” and adaptation. The development of artificial systems of collective robotics represents opportunities to surpass these limits. The present system offers numerous insights into optimizing these complex processes.

The Nanorobotic Environment

The nano domain, which is a billionth of a meter, is measured in millionths of a meter. A single oxygen atom is roughly a single nanometer across. A micron is a millionth of a meter. The width of a human hair is about 60,000 nanometers.

The present invention focuses on the synthetic development of objects that are in a middle (meso-nano) sphere somewhat between the atomic size (micro-nano) of simple atoms and the mega-nano domain of micron-sized objects. While it is true that scientists have built, from the ground up, that is, atom by atom, objects such as elegant geodesic nanotubes made of carbon atoms, objects in this domain are too small and too expensive to construct to be useful for an active intelligent system. In order to be useful, a nanorobotic system requires numerous and economical robots dependent on mass production techniques that must generally be considered from the perspective of a top down strategy, that is, by utilization of largely lithographic procedures.

The nanorobotic entities described herein generally consist of objects with dimensions from 100 nm to 1000 nm (1 micron) cubed, but can be smaller than 100 nm or larger than ten microns. This size is relatively large by nanotechnology standards, but is crucial in order to maintain functionality. Keep in mind that a white blood cell is comprised of about 100,000 molecules and fits into this meso-nano domain. The micron-scale space of inter-object interaction may be comprehended by analogy to a warehouse in which nanoscale objects interact. In order to be useful, nanorobots require complex apparatus that includes computation, communications, sensors, actuators, power source and specific functionality, all of which apparatus requires spatial extension. While this domain specification is larger than some of the atomic-scale research in nanotechnology, it is far smaller than most microelectronics,

While the larger meso-nano assemblies described herein possess a specific geometric dimensionality, the size dimensions of the domains in which they operate are also critical to consider. In these cases, each application has a different set of specifications. In the case of the human body, specific cells will have a dimensionality that is substantially larger than the complex molecular-size proteins that are constructed for interoperation within them.

Over time, however, it will be possible to make very small, useful micro-nano scale robots for use in intelligent systems. Thus, we may conceive of several generations of scale for these systems, the first being in the meso-nano domain.

### Synthetic Biology

An emerging field of synthetic biology manipulates combinations of transformable organic components. By using human intervention to alter organic biological parts in new ways, synthetic biology assembles and reassembles organic parts in unnatural ways, thereby producing artificial Darwinian systems that supplement biological systems. As an example of this new science scientists have combined organic material in new ways to create an artificial synthesis of new bacterial and viral organisms. Similarly, the toxicity of a virus may be inactivated by modifying its DNA using recombinant techniques.

Rather than modifying the parts of an organism's DNA, synthetic biology seeks to engineer an entirely new species by custom engineering the organism's whole DNA. Synthetic biology uses biomemetic chemistry to synthesize organic molecules to emulate biological behaviors. This approach to creating new life combines DNA and RNA parts from raw amino acids to create novel genetic structures. These genetic configurations are reverse engineered by observing specific natural protein behaviors created from specific gene sequences by using gene targeting techniques. Natural proteins with regular behaviors and expected functional consequences are engineered from specific customized genetic sequences.

Schafmeister performs research at the University of Pittsburgh on synthetic proteins. Small molecule ligands bind to proteins to modify proteomic functions. He developed new small molecule ligands shaped as flat disks. Small molecule ligands bind to protein surfaces to disrupt protein-protein interactions. By blocking some protein functions, it is possible to test protein operations, which is useful in identifying protein function. In this process, synthetic biology is used to design and develop artificial organic proteins.

The work of Benenson and Shapiro at Harvard develops synthetic biology to organize an autonomous molecular computer that performs specific cellular functions. Each cell is a computer in the sense that proteins transfer information by interacting with each other. The biomolecular computer diagnoses disease and administers a drug on demand when the disease is encountered. This process is organized by inserting genetic material into DNA that tests the effects of a specific gene. Once specific cellular states and inputs are detected, the cell is programmed to respond. For instance, if a genetic dysfunction is detected by identifying a specific biomarker, a specific chemical is activated to control the dysfunction.

### SUMMARY OF THE INVENTION

One of the exciting application categories of nanorobotics is medicine. Unlike intracellular biological applications, medical applications of nanorobotics involve targeting a particular medical problem. Several medical categories present major problems for which nanorobotics provide solutions. These problems include cardio-vascular health, immune system function, cancer and diabetes. In addition, medical application categories address problems involving drug delivery mechanisms and diagnostics.

### INNOVATIONS, APPLICATIONS AND ADVANTAGES

Regarding medical applications, the present system allows drugs to be delivered, and regulated, more effectively to precise targets. These processes are useful for cardio-vascular applications as well as in treating diabetes. These processes also apply to intracellular cancer therapies.

There are numerous applications of the present system to repair specific medical conditions. CNRs are useful to cauterize wounds in patients with emergency trauma.

CNRs are applied to nerve cells to block pain signals. This process occurs because CNRs configure into synthetic molecules that are activated and modulated to control pain from specific nerve fragments.

Because of their malleability capabilities, CNRs are very useful in dental applications as well, particularly in repairing enamel and nerve damage and to stop bleeding.

CNRs are useful for neural disorders. Primarily because of their ability to penetrate cellular mechanisms, CNRs are useful in neurosurgery procedures that would be otherwise inaccessible. CNRs interoperate in hitherto impenetrable intracranial environments, perform a function and are then extracted. Specifically, CNRs are useful in order to perform complex regulatory functions that involve feedback in dynamic neural processes.

CNRs are also useful in dermatological applications. Though in this application, CNRs are used to defy the appearance of aging, these processes exploit self-repairing cellular functions.

Finally, CNRs are useful to accelerate cellular regeneration processes in order to promote healing. This function is performed by accelerating the operations of proteins and enzymes in affected tissues. CNRs are targeted specifically at regenerating cells, thereby increasing efficiency.

### DESCRIPTION OF THE INVENTION

(1) Mapping the Body using CNRs

While there are different ways of tracking CNRs, including using tags in individual nanorobots that behave as beacons in order to identify their specific locations and progress, the CNR system maps the architecture of an organism (such as the human body) on the molecular level. Though the precise detail of the map depends on the specific CNR mission, the CNRs explore specific tissue and cell types.

The CNRs congregate at specific tissue sites and await information about mission parameters with new goals. The CNR teams cluster at a specific location before performing an organized function to solve a key problem and then return to the location when the mission is completed.

The CNRs are also used to target and mark specific cells. This is useful in identifying specific molecular locations in order to engage future CNR teams to perform a function. For example, pathological cells that result from a mutation or combination of mutations may be targeted, marked and then attacked, thereby emulating the immune system as it identifies, marks and attacks a neoplasty.

The mapping process employed by the CNR teams also traces pathways of functional behaviors within intercellular mechanisms. The CNRs map cellular differentiation and compare the results of a particular mapping sequence with the general human anatomy and physiology map to identify aberrations.

The mapping process begins with the CNR team placed in a specific location. The collective then breaks into clusters and migrates to specific locations by using various mobility patterns, while the mapping process is recorded. Since each cell type is like a separate country, each cell type must be evaluated separately. In particular, the aging process yields differences in conditions of cells in various tissues from among specific organ systems.

The CNRs also work together in an integrative system with external computation resources. The initial data in a map created by CNRs is transmitted to external computation for detailed analysis and organization. The external computer analysis guides the CNRs to specific cell types and to dysfunctional cells. In addition, CNRs use the specific maps of each individual created by DNA analysis in order to coordinate specific intracellular functions.

After they have mapped the tissue, the CNRs are activiated to perform a specific function in order to meet a goal or solve a problem. In order to meet goals, the CNRs use nano evolvable hardware (N-EHW) mechanisms to transform into an active mode in order to solve problems by interacting with, and adapting to, the evolving environment.

(2) Collective Nanorobotic System for Cardio-Vascular System for Regulation of Arterial Plaque and Nanobacteria

Risks for heart disease include high levels of LDL cholesterol and cardio reactive protein (CRP) because these contribute to arterial plaque and nanobacteria that ultimately clog arteries. While high LDL is a predictor of increased risk for cardiac trauma, high HDL cholesterol, which is comprised of small particles which remove the large particle LDL, is beneficial for reducing arterial plaque. Statins decrease LDL levels by affecting enzymes in the liver, though their use is not without risks or side effects.

CNRs are useful in reducing arterial plaque in several ways. First, CNRs identify plaque deposits in the arterial pathways. The CNRs ride the currents of the blood stream and map out the arterial system with great detail. This process is primarily diagnostic; it produces maps that rank priorities to address. Second, the nanorobotic collectives actively intervene by delivering drugs to specific locations. Third, the CNRs themselves behave as HDL cholesterol and abrasively remove deposits of LDL. The CNRs continually report to an external computer with diagnostic feedback on their progress toward achieving their goal of reducing plaque. These feedback mechanisms are modified by the physician or surgeon.

This system is useful in the operating room in order to monitor the progress of administered CNRs. The CNRs then actively deliver chemicals to particular locations within the arterial pathways, in particular by targeting specific high density plaque deposits. Because they act deliberately, the CNRs prioritize their missions and attack the most important spots. In particular, the CNRs may apply proteins that block or complement the high CRP levels to reduce their adverse effects. The CNRs remove the plaque deposits without flushing them in the system. Rather, they absorb the waste of the plaque and carry it out of the system to prevent exposing the cardiac system to a sudden build up in toxins. After the procedure, the CNRs are extracted, sterilized and reused.

Thus CNRs provide a useful way to regulate the optimal cholesterol and CRP levels of patients without interventions that may provide toxic side effects.

(3) Collective Nanorobotic System for Insulin Regulation

The pancreas produces insulin for proper regulation of glucose in the blood stream. This glycation regulation process is critical to the healthy operation of cellular processes. CNRs are useful in several complex processes related to pancreatic function.

The main insulin-secreting part of the pancreas, which is part of the endocrine system, is the isles of Langerhans. The isles of Langerhans have about a million islets in a healthy adult human pancreas; each islet contains about 1000 cells that are structured in clusters. The pancreatic isles use a mechanism of amyloidogenesis to create amyloid polypeptides.

The isles create four main types of cells. Beta cells (65-80%) produce insulin. Alpha cells (15-20%) produce and inhibit glucagen, which is an opposing hormone that releases glucose from the liver and fatty acids from fat tissue. Delta cells (3-10%) produce somatostatin, which inhibits somatotropin (a pituitary hormone), insulin and glucagons. Finally, pancreatic polypeptide (PP) cells (1%) secrete polypeptides which suppress pancreatic secretion and stimulate gastric secretion.

Insulin activates beta cells and inhibits alpha cells. Glucagon activates beta cells and delta cells. Somatostatin inhibits alpha cells and beta cells. The constellation of processes embodied in these cell types creates the paracrine feedback system of the islets of Langerhans. The self-organizing system uses paracrine and autocrine communication between the islets. The autocrine process sends signals to the same cell by secreting a chemical messenger. The paracrine process sends signals to cells next to the cell. For instance, beta cells are only linked to other beta cells in this chemical communication system.

The process of insulin production to regulate the body's glycation process is critical for healthy cellular functioning. When too much fat and carbohydrate are in the diet, the pancreas is forced to produce more insulin to regulate the high intake levels. The result is an increase in the storage of fat, which ultimately manifests as obesity. The pancreas of obese patients is taxed until it ultimately is unable to produce insulin. The patient develops (type II) diabetes and requires tight regulation of blood sugar by regular insulin injections.

CNRs are useful in several respects to regulating insulin. First, CNRs go beyond the limited autocrine and paracrine pancreatic mechanisms of chemical communication. These biological processes use nearest neighbor communication models for specific cell types in the isles of Langerhans. However, CNRs are able to communicate throughout the region to provide regulated mechanisms beyond merely the nearest neighbor capability of natural processes. Second, in the case of dysfunctional pancreatic behaviors, CNRs emulate the proper functioning of the isles of Langerhans. The CNRs conduct a glycation process of treating blood sugar with insulin in a similar fashion to the operation of yeast, which conducts a process of converting sugar in juice to alcohol. Third, the CNRs can be organized into an artificial implantable device that emulates the functioning of a pancreas. The self-regulating pump is constructed of CNRs that are organized to emulate the specific functions of the isles of Langerhans. In an alternative embodiment, the pancreatic-like device is external and wearable.

In addition to the development of an artificial pancreas with CNRs, other artificial organs, notably the liver, kidney, spleen or eye are organized to perform specific artificial functions by employing CNRs. There are further applications in which nanorobotics plays a supporting role in complex artificial organs that consist of implantable electro-mechanical devices. Complex arterial and nerve pathways are able to be constructed from CNRs, while the traditional functional mechanism is constructed of a traditional mechanical apparatus. The CNR communication and sensor sub-systems provide greater flexibility than a biological system.

(4) Collective Nanorobotic System for Drug Delivery with Feedback Mechanism

CNRs are used to deliver nanocargoes, particularly chemicals directly to targeted cells. The nanorobots that carry cargoes have a specific structure that includes a double insulated device with an inner hull to hold chemicals and a hydrophobic surface to penetrate cell membranes. The cargo nanorobots are made of flexible materials so as to penetrate a cell membrane without creating a destructive reaction. In one embodiment, the outer shell, which is doped to prevent immune response, dissolves after cellular penetration, while the active robot operates within a cell.

In some cases, nanotubes act as structures that supply fluid to an active nano-pump that then fills up mobile nanocontainers. In a functional system, multiple nanorobots with nanocontainers act as messengers to bring chemicals to targets and return to a remote location to obtain more chemicals and repeat the process until a task is completed.

This system is useful for supplying highly targeted proteins to a cell. Personalized medicines that are designed to cure a specific genetic disease caused by a patient's unique combinations of genetic mutations are delivered to highly targeted cells, such as tumor cells, using collectives of cargo nanorobots.

The problem of nanorobotic mobility is solved by using physical properties that exploit the natural fluidic nature of intracellular systems. One solution to the problem of delivering CNRs to a site is to use monoclonal antibodies as vehicles to identify and target a particular tissue location that attracts the antibodies.

The present system also uses micro-scale modules that are under pressure in order to initiate controlled bursts of pressurization so as to activate CNR clusters to deliver the nanorobots to a particular location. These micro-capsules carry the CNR teams and disgorge the CNRs selectively on demand. The CNRs then perform a function and return to the micro-capsule base, for example, to get spare parts in order to perform N-EHW functions.

In another embodiment of the present system, a stent is surgically installed in a patient and acts as a platform for the launch of CNRs. The CNRs perform a function and then return to the stent when the task is completed. After a procedure, the stent may then be surgically removed. Other modules may be implanted to accomplish the same task.

In still another embodiment of the invention, CNRs are used to identify, target and deliver radioactive elements in order to attack a tumor. The system is particularly suited to addressing the problem of killing tumors that are too small for detection or too remote for surgical intervention.

The system is also useful for detecting the presence of chemicals in specific tissues. CNRs add or subtract chemicals from specific cells on-demand. Once CNRs assess the decline in specific chemicals, they add or remove other chemicals in performing a specific procedure.

In this way, the CNR system modulates, or regulates, cells, much like a pace-maker regulates a heart's functioning. The CNRs patrol the body by using the mapping system, identify problems and anomalies, and call up reinforcement specialists when needed to solve problems. In some cases, the CNRs uses N-EHW transformational processes in order to solve problems in real time by converting from a passive identification system to an active interventionist system.

This system is useful when combined with surgical procedures. The CNRs help to identify and target particular cells. In addition, once an intervention has been performed, the CNRs help the tissue heal more quickly by delivering chemicals directly to the tissue.

The advantage of the use of this system is that the application of chemicals is modulated by feedback processes. Hence, drugs are not merely delivered but automatically and continuously monitored as well. In addition, the system allows the intra-cellular application of chemicals.

The system allows for the identification of a problem, identification of the specific chemical needed to solve the problem, the obtaining of a needed chemical from a remote location, the delivery of the chemical and the continuous assessment of the problem and the solution. This delivery system using CNRs provides self-organization via regulatory and feedback mechanisms.

In another embodiment of the system, proteins and antibodies are themselves used to deliver CNRs to specific locations since their behaviors are generally predictable.

In one use of the delivery process of CNRs of the present system, cancer cells are targeted for delivery of specific substances at particular locations. The cellular problem is identified, the cells are penetrated by cargo nanorobots, the cell nucleus is identified and penetrated and specific chromosomes in the DNA are identified. A mutated gene is then identified, and a synthetic procedure of constructing and applying unique CNR configurations is used to repair the gene.

At the end of the delivery process, cargo nanorobots are collected, accumulated and extracted at regular intervals, particularly as the mission is completed or the chemical cargoes are depleted.

(5) Collective Nanorobotic System for Stent

CNRs are useful for other medical instrument applications, particularly, stents that are used to support blocked arteries or veins. Stents comprised of an outer layer consisting of CNRs allow for increased effectiveness. CNRs are used on stents to activate other processes. The stents also serve as locations from which to launch specific CNR missions by utilizing an accessible compartment on the stent surface.

One of the problems with existing stent technologies is that the stents are passive and fixed in size and configuration. However, with CNRs, stents are adaptive, modular and flexible in configuration, modifying their structure to the needs of the patient's problem. In this capacity, interactive stents comprised of CNRs behave as system regulators. Because they are doped with chemicals or proteins, CNR-enriched stents are proactive in identifying and solving problems.

In an important application of CNRs to stents, the CNRs are used to treat strokes. After a stent is placed in a patient's carotid artery, the CNRs monitor blood flow before a stroke. In the case of ischemic stroke, CNRs move on the inside of a blood vessel to clear obstructions by burrowing in the center of the obstruction. In the case of hemorrhagic stroke, CNRs rapidly plug a hole in an artery to repair it until surgical intervention is made. These procedures are implemented before an event by implementing in vulnerable patients with a history of stroke. The use of CNRs are also applied after a stroke event to rapidly stabilize a patient until surgical intervention is possible.

In one embodiment of the present system, stents are used as launching pads for CNR teams to perform specific functions and then return to the stent upon program completion. In this sense, CNRs are released from stents on-demand to solve specific biomedical problems, such as removing occlusions (e.g., blood clots). The CNRs are contained in compartment within the stent and have access to the stent's outer membrane via a hole in the stent that contains a valve. CNRs obtain chemicals from a reservoir in the stent, perform a delivery function and return for more chemicals until the reservoir is depleted. The chemical reservoir is surgically refilled periodically with endoscopic techniques. In this way, the CNRs behave as a time-released team that responds to specific problems in waves as they are required. In this sense, advanced stents behave as fixed platforms that perform multiple functions by combining both chemicals and CNRs.

In another embodiment, CNRs are used to filter chemicals, cells, proteins and antigens from a position on a stent. The CNRs form a layer on a stent to pick out objects in the blood stream. In particular, CNRs in a stent are useful to selectively filter methyl molecules that regulate genetic behaviors.

(6) Diagnostic System using Collectives of Nanorobots

CNRs are useful in diagnostics. CNRs are used to detect cellular neoplasms. They are also used to detect the presence or absence of a protein. In order to detect an object, CNRs employ sensors and probes that use network communication functions to relay information to other nanorobots and external computers.

Unlike typical passive diagnostic apparatuses, however, CNRs provide real-time feedback mechanisms that modulate chemical applications. In other words, because the system allows for social intelligence, and self-organization, in its application as a diagnostic system, the CNRs integrate diagnostic functionality with active functionality to solve problems. The advantage is that once the CNRs are in the body, the can actively perform a positive function as well as the initial diagnostics.

CNRs are used as taggants for diagnostics. Tissue is tagged by nanorobots and the cellular performance is tracked because the tags are active and “intelligent.” Multiple tags work together in this system to coordinate behavior. The targets' data are then transmitted via nanorobotic communications to update diagnostics.

(7) CNR Applications to Blocking Metastatic Cancers

Cancer mortality is generally caused by the metastatic processes of spreading cancer cells from one tissue type to other tissues. In order to limit the mortality from this disease after it has been detected, it is critical to prevent its spread. In particular, specific types of cancers tend to spread to specific tissue types. For instance, breast cancers tend to spread initially to the bone and lung. Carcinomas will tend to spread to the brain. The lungs, liver and brain tend to be recipients of a range of metastases in part because of their strategic locations and integral access to the blood stream.

CNRs are used to identify the metastases of various cancers and then to block them. The CNRs identify cancer cells from one tissue that have spread to other tissues and destroy them by engulfing or rupturing them.

One way to optimize the use of CNRs in order to limit the spread of specific cancers is for CNRs to patrol particularly risky tissues, such as lungs, once a breast cancer is detected. The CNRs then embargo specific cells.

When a metastatic cancer cell is detected, CNRs are combined together to perform specific operations that emulate phagocytes in the human immune system. The information about the metastases is then provided to a database in order to indicate problem cells for which to detect in the future. This process assists future detection procedures and increases the speed of targeting.

(8) Collective Nanorobotic System for Destroying Tumor Cells

Since tumors are best treated when they are small, CNRs provide a mechanism to identify cancer early by using diagnostic capabilities. Once identified, CNRs emulate the killer T cell functions by initiating respiratory death of tumor cells. This is accomplished through penetration of cell membranes with large holes that allow liquids and ions to pass through and destroy the cell.

The process of destroying tumor cells begins by CNRs targeting the outer layer of the cluster of dysfunctional cells. After initially attacking the outer cell layer, the CNRs make multiple passes until the task of killing the problem cells is completed.

Since neoplasms are recognizable from healthy cell growth, CNRs are useful to identifying these problem cells. CNRs then target only the narrow band of tumor cells and leave the surrounding cells alone to flourish.

Reference to the remaining portions of the specification, including the drawings and claims, will realize other features and advantages of the present invention. Further features and advantages of the present invention, as well as the structure and operation of various embodiments of the present invention, are described in detail below with respect to accompanying drawings.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes in their entirety.

### DESCRIPTION OF THE DRAWINGS

FIG. 1 is a diagram showing the use of collectives of nanorobots (CNRs) to penetrate tissue.

FIG. 2 is a schematic diagram showing the pattern of movement of CNRs through different tissues.

FIG. 3 is a diagram showing the penetration of a cell by CNRs.

FIG. 4 is a flow chart describing the process of using CNRs to perform a function in tissue.

FIG. 5 is a schematic diagram showing CNRs used to identify blocked arteries.

FIG. 6 is a schematic diagram showing CNRs removing arterial blockage.

FIG. 7 is a flow chart describing the process of using CNRs to remove arterial blockage.

FIG. 8 is a diagram showing an artificial pancreas composed of CNRs.

FIG. 9 is a flow chart showing the use of CNRs to modulate blood sugar in an artificial pancreas.

FIG. 10 is a diagram describing a cargo nanorobot.

FIG. 11 is a diagram showing the use of multiple cargo nanorobots to obtain chemicals from a central chemical depot.

FIG. 12 is a diagram showing the operation of cargo nanorobots from a chemical depot to a cell.

FIG. 13 is a diagram showing an antibody carrying a CNR into a cell.

FIG. 14 is a schematic diagram showing cargo nanorobots in an arterial system.

FIG. 15 is a diagram showing a pressurized microcapsule delivering CNRs in an arterial system.

FIG. 16 is a schematic diagram showing CNRs attacking a tumor using radioactive chemicals.

FIG. 17 is a schematic diagram showing a stent with compartments containing CNRs which perform functions in the arterial system.

FIG. 18 is a diagram showing the process of using nanorobots to tag cells.

FIG. 19 is a diagram showing the use of nanorobots to block the metasticization of cancer cells.

FIG. 20 is a flow chart describing the process of using CNRs to block the metasticization of cancer cells.

FIG. 21 is a diagram showing the use of nanorobots to penetrate a cell.

FIG. 22 is a multi-phasal diagram showing the use of nanorobots to destroy a cell.

### DETAILED DESCRIPTION OF THE DRAWINGS

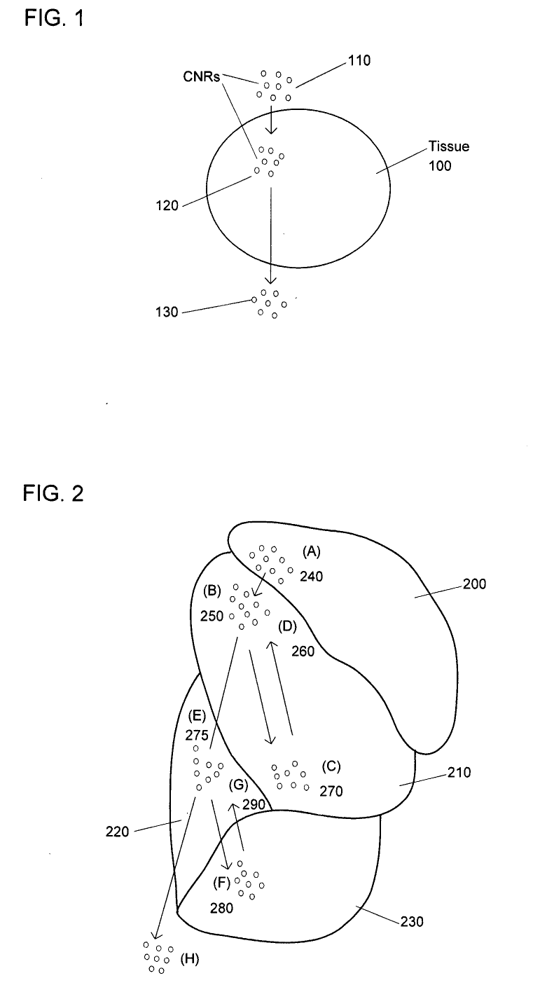

FIG. 1 shows collectives of nanorobots (CNRs) (110) entering tissue (100), moving through the tissue (120) and exiting the tissue (130). FIG. 2 shows CNRs moving initially (240) from one tissue (200) at position A to an adjacent tissue (210) at position B (250). The CNR then moves to position C (270) and back to position D (260) in the same tissue (210). The CNR then moves to another adjacent tissue (220) at E (275) and further moves to an adjacent tissue (230) at F (280). The CNR returns to adjacent tissue (220) at G (290) and, finally, out of the tissue to position H.

Recording the data from the journey through the tissue allows the CNRs to map the tissue. The data is then transferred to an external database for analysis. This data capture and analysis allows customized mapping of an individual's body. As conditions in the patient change, a new set of data is captured and the data sets are compared in order to assess the degradation. Use of nanorobotics to map the body is particularly useful for assessing cellular and molecular changes to complement radiological exploratory techniques.

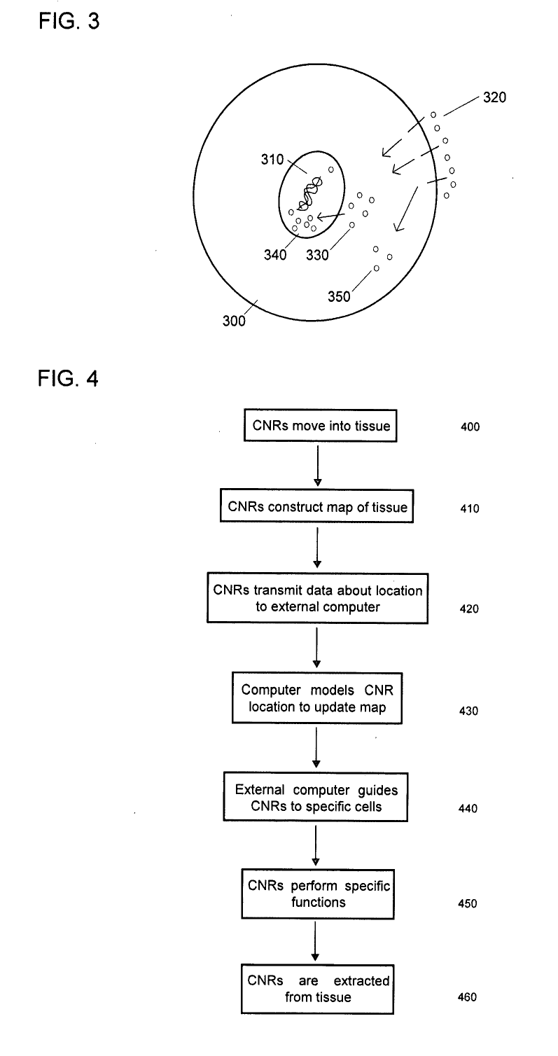

FIG. 3 shows a representation of CNRs (320) entering the outer membrane of a cell (300), where it divides into several groups (330 and 350). The CNR (340) then enters the cell nucleus (310).

FIG. 4 describes the process of CNRs penetrating tissue. After the CNRs move into tissue (400), they construct a map of the tissue (410). The CNRs transmit data about their location to an external computer (420). The external computer models the CNR location to update the map (430) and guides the CNRs to specific cells (440). The CNRs perform specific functions (450) and are extracted from tissue (460).

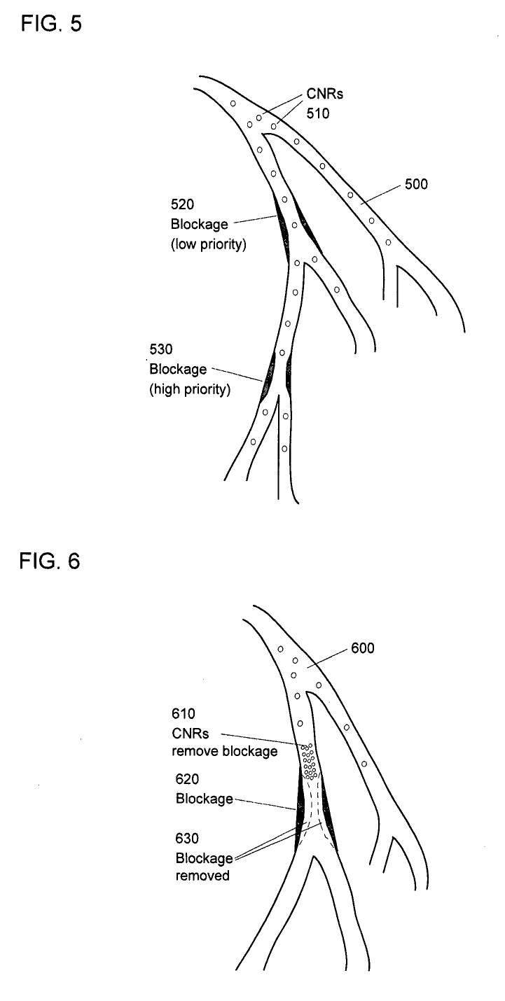

FIG. 5 shows the use of CNRs to identify arterial blockage in an arterial system. The CNRs monitor the condition of the arteries and survey the relative priorities of blockage. The CNRs (510) identify the low priority blockage (520) and the high priority blockage (530), while they observe the healthy arterial functioning (500) of other areas. This process of surveying the various areas of arterial blockage is important in order to establish the priority of removing the blockage.

FIG. 6 shows how CNRs are used to remove arterial blockage. The blockage (620) is removed by applying the CNRs to abrasively attach to the inner artery wall to gradually remove inflammation and debris until blockage is removed (630). In another embodiment, the CNRs administer a drug directly to the blockage until excess blockage is removed.

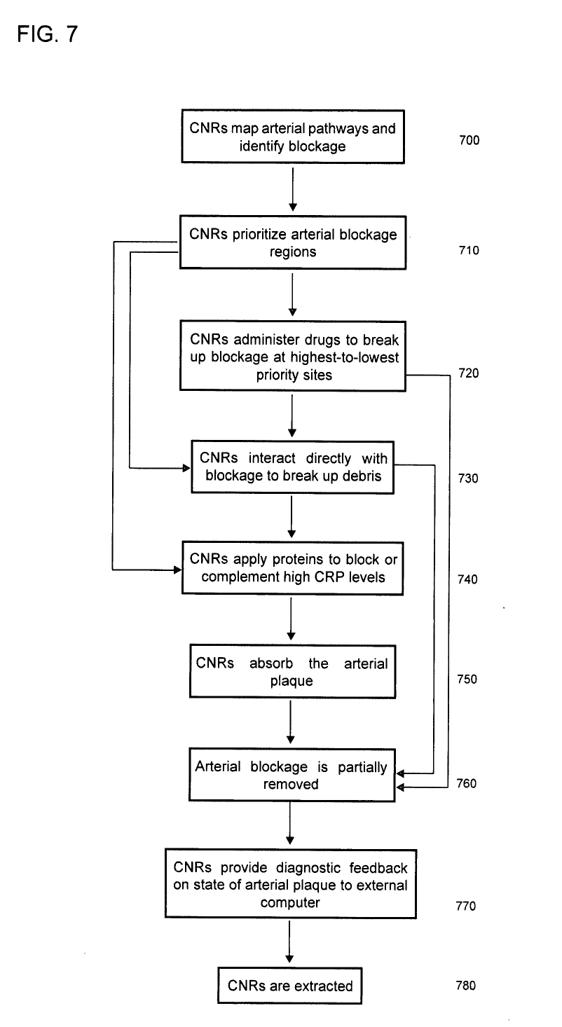

FIG. 7 shows how the CNRs are applied to remove arterial blockage. After the CNRs map arterial pathways and identify blockage (700), they prioritize the arterial blockage regions (710) according to the greater blockage. The CNRs administer drugs to break up blockage at the highest-to-lowest priority sites (720). Alternatively, the CNRs interact directly with blockage to break up debris (730). In another alternate option, the CNRs apply proteins to block or complement high cardio reactive protein (CRP) levels that are causing the blockage (740). Regardless of the main method used to disrupt the blockage, the CNRs absorb the arterial plaque generated by removing the blockage (750). The arterial blockage is partially removed (760) and the CNRs provide diagnostic feedback on the state of arterial plaque to an external computer (770). The CNRs are then extracted from the patient (780).

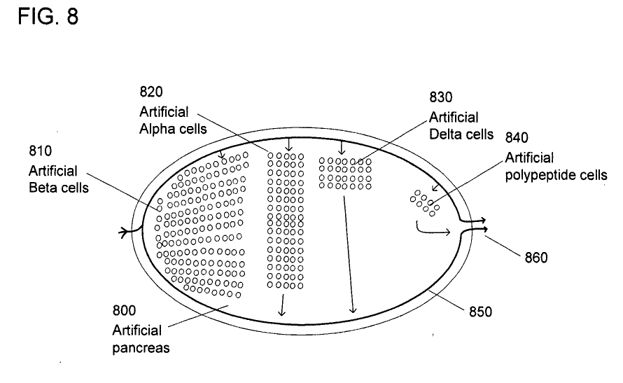

FIG. 8 shows an artificial pancreas composed of CNRs. The CNRs emulate the function of Alpha cells (820), Beta cells (810), Delta cells (830) and polypeptide cells (840) in the artificial pancreas (800). After a patient's blood is input into the device, it is assessed for insulin levels. The artificial pancreas uses the CNRs to modulate the use of different levels of insulin (Beta cells), glucagon (Alpha cells), somatastatin (Delta cells) and polypeptides (polypeptide cells). Once the blood enters the device (on the left in the diagram), the blood sugar is measured and the artificial Beta cells provide insulin to modulate the equilibrium of the blood sugar. The blood is then passed to the artificial Alpha cells, the artificial Delta cells and then the artificial polypeptide cells, which modulate the application of chemicals to treat the blood sugar. At each stage, the blood flows away from the artificial cells to be remixed by the appropriate chemical. Once the correct levels of blood sugar are achieved, the blood moves down the device at the inner lining (850) to exit (860).

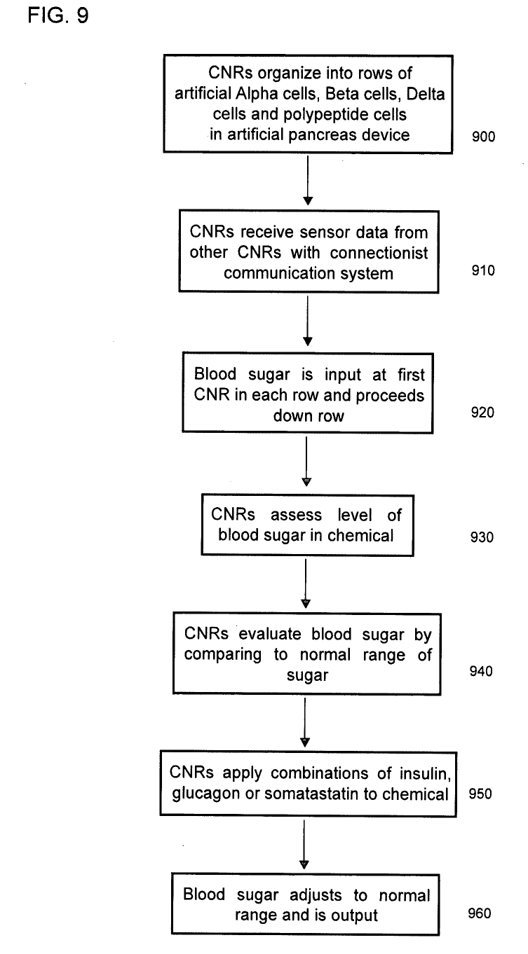

FIG. 9 describes the process of using CNRs in an artificial pancreas. The CNRs first organize into rows of artificial Alpha, Beta, Delta and polypeptide cells in the artificial pancreas device (900). The CNRs receive sensor data from other CNRs by using a connectionist communication system (910). The blood sugar is input at the first CNR in each row and proceeds down the row (920). The CNRs assess the level of sugar in the blood (930) and evaluate blood sugar by comparing it to a normal range of sugar (940). The CNRs apply combinations of insulin, glucagon, somatastatin or polypeptides to the chemical composition (950) and the blood sugar adjusts to a normal range and is output from the device (960).

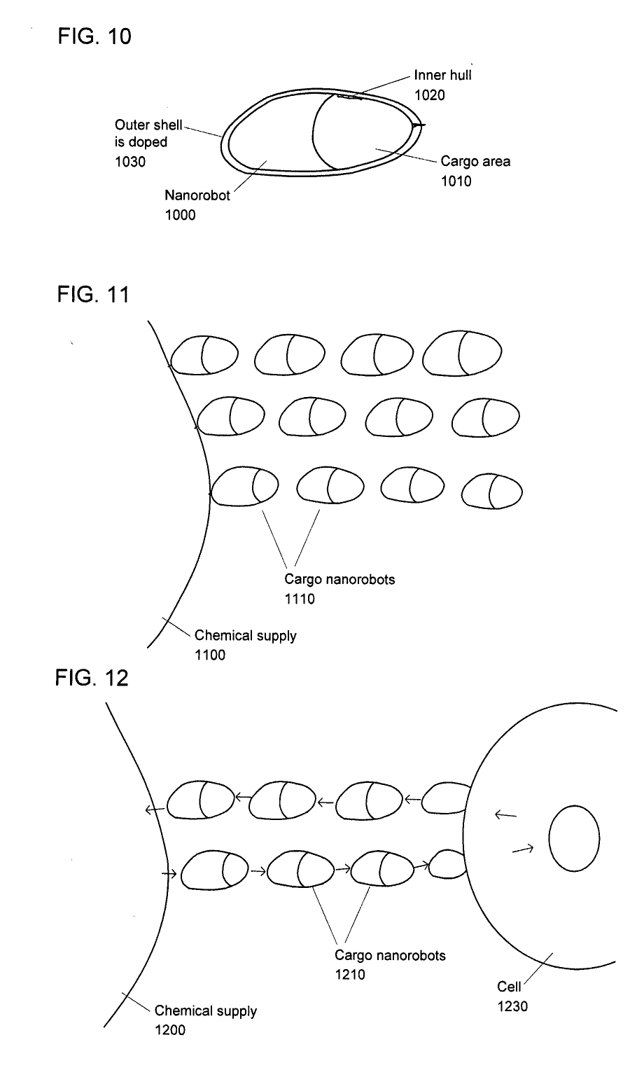

FIG. 10 shows a cargo nanorobot which is used to carry chemicals within tissues. The cargo nanorobot (1000) has an outer doped shell (1030) in order to penetrate tissue without eliciting an immune response. The inner hull (1020) of the cargo nanorobot is used to insulate the chemical cargo from other sections of the device. The cargo area (1010) is clearly specified.

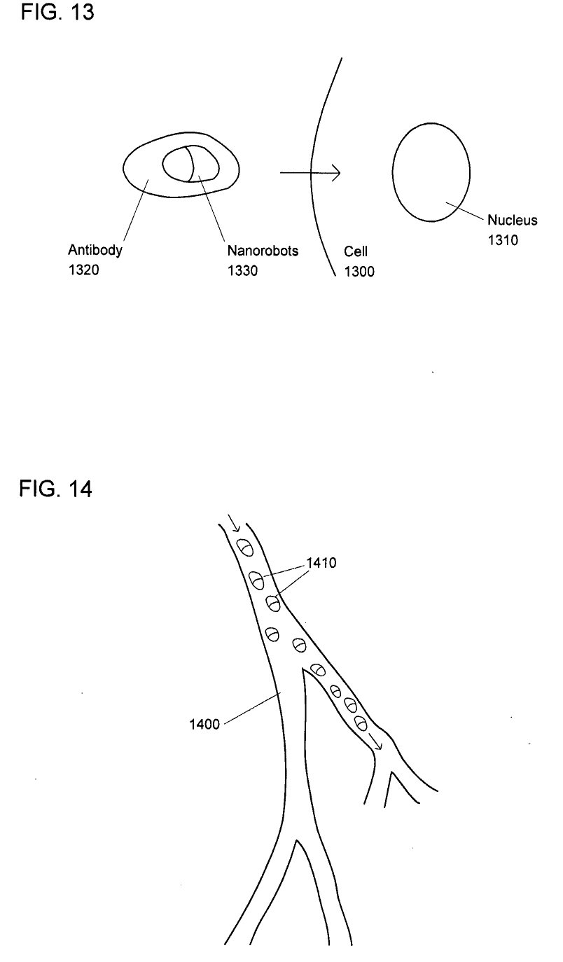

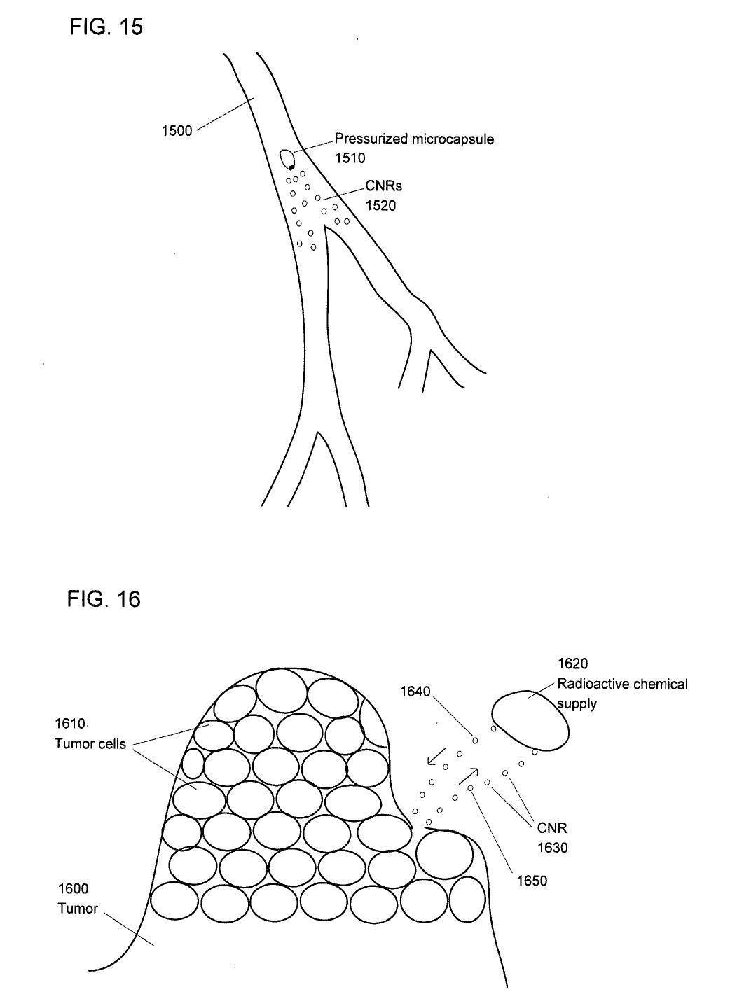

In FIG. 11, cargo nanorobots (1110) are shown in rows as they are connected to a chemical supply (1100). FIG. 12 shows the cargo nanorobots (1210) as they move from the chemical supply (1200) to a cell (1230) and then back to the chemical supply. FIG. 13 shows an antibody (1320) carrying nanorobots (1330) into a cell (1300). FIG. 14 shows cargo nanorobots (1410) in an arterial system to carry chemicals to specific targeted locations to solve arterial blockage problems or to carry CNRs to cells. FIG. 15 shows a pressurized microcapsule (1510) disgorging CNRs (1520) in an arterial system (1500).

FIG. 16 shows a tumor (1600) that is attacked by CNRs (1640) as they administer a radioactive chemical from a chemical supply near the tumor (1620) to specific tumor cells (1610). The CNRs move into and out of the tissue to return to obtain more radioactive chemicals until the tumor is killed.

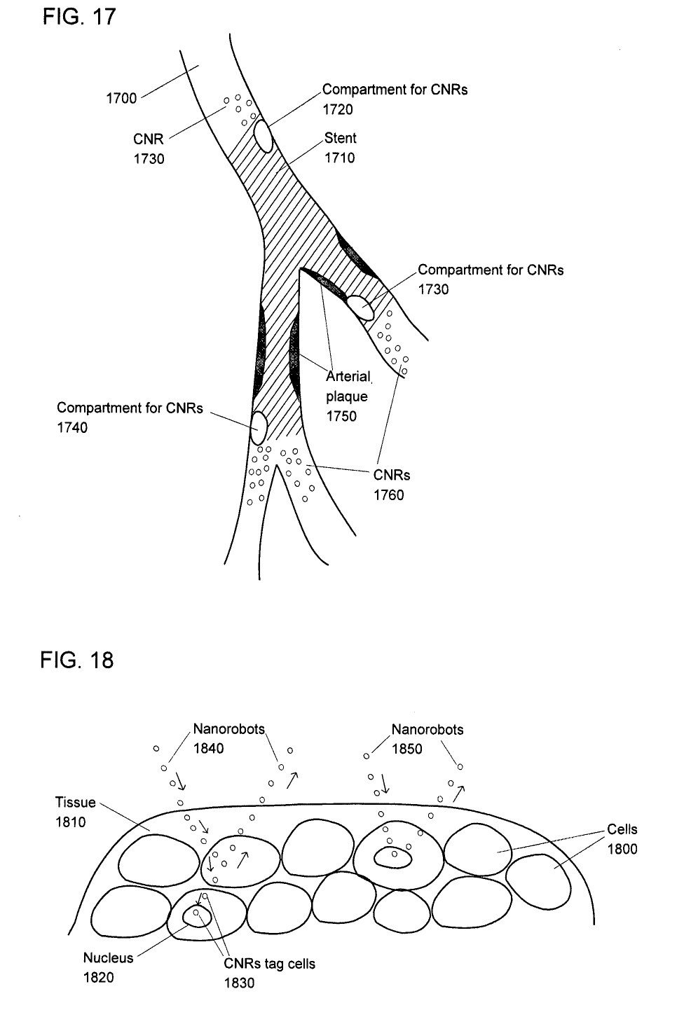

FIG. 17 shows the use of CNRs in pockets installed in a stent. The wire mesh stent (1710) is placed in the arteries as shown (1700). The CNR compartments (1720, 1730 and 1740) are used to house the CNRs for specific missions. Specifically, the first compartment (1720) is used to collect incoming CNRs as they return from specific missions in the blood stream. The other compartments (1730 and 1740) are used to administer CNRs for different purposes. In the case of the compartment at 1730, the CNRs will address the problem of arterial plaque in the ways described above.

FIG. 18 shows the use of CNRs to penetrate tissue to tag cells. The CNRs (1840 and 1850) enter the tissue (1810) to identify specific cells. In the example, they approach a cell and tag the cell (1830) and its nucleus (1820). Once tagged, the CNRs depart the cells and the tissue. This process is useful in targeting cancer cells for later delivery of specific chemicals to kill the cells. In other cases, targeting is useful to track the behavior of specific cell types.

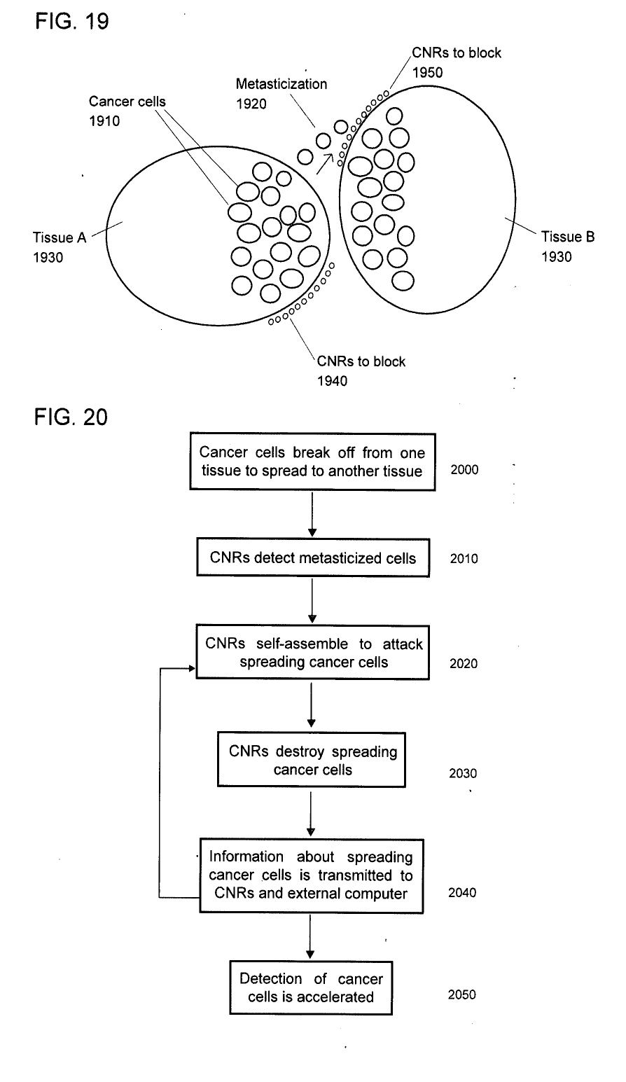

FIG. 19 shows the use of CNRs to control metasticization. The cancer cells (1910) at tissue A (1930) are identified and blockaded by a group of CNRs (1940). As the cancer cells spread to another tissue (1930), the CNRs blockage these cells by providing a layer of protection (1950).

FIG. 20 shows the process of using CNRs to limit metasticization. After cancer cells break off from one tissue to spread to another tissue (2000), the CNRs detect the metasticized cells (2010). The CNRs self-assemble to attack (2020) and destroy (2303) the spreading cancer cells. Information about the spreading cancer cells is transmitted to CNRs and the external computer (2040) and the detection of the cancer cells is accelerated (2050).

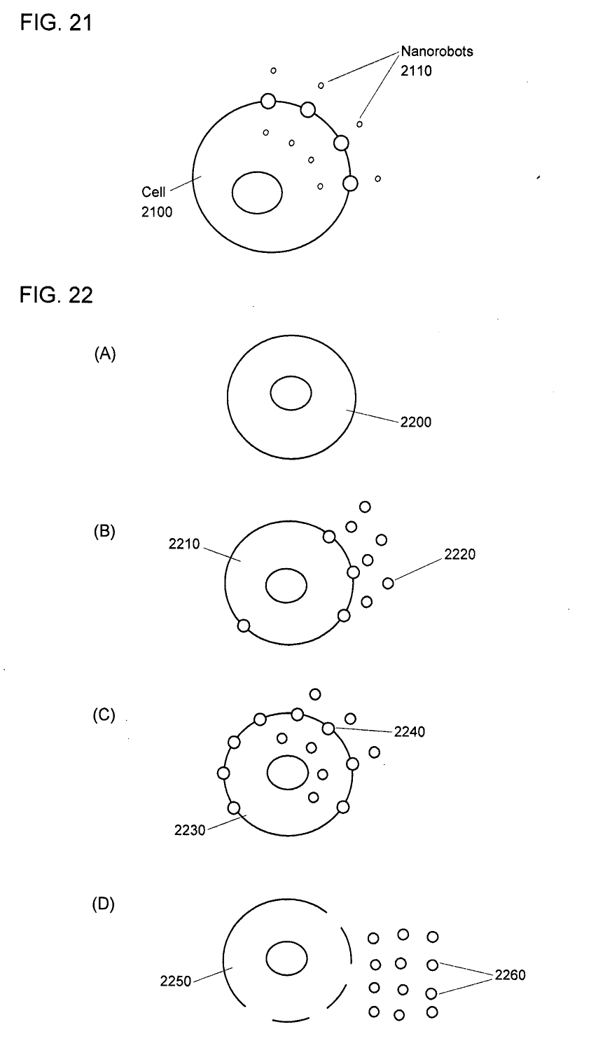

FIG. 21 shows nanorobots (2110) attacking a cell (2100) that is either cancerous or infected with antigens. In FIG. 22, the process of using CNRs to attack cells is further delineated. At phase A, the cell (2200) is identified as being infected or cancerous. At phase B, CNRs (2220) commence an attack on the cell (2210). At phase C, the CNRs engulf (2240) the cell (2230), after which the cell bursts and dies. At phase D, the CNRs are extracted (2260) from the remains (2250) of the cell.

What is claimed is:

1. A system for managing automated collective nanorobots (CNRs), comprising:
A plurality of nanorobots, each nanorobot including program code configured to communicate and exchange information with other nanorobots;
Wherein CNRs are injected into a patient;
Wherein the CNRs congregate at specific tissue sites to await information about mission parameters with new goals;
Wherein the CNRs target and mark specific cells with tags for eventual intervention;
Wherein the CNRs map cellular differentiation and compare the results of a particular mapping sequence with general human anatomy and physiology maps to identify aberrations of a particular patient;
Wherein the CNRs migrate to specific cellular locations while the mapping process is recorded;
Wherein the CNRs transmit mapping data to an external computer; and
Wherein the external computer analyzes the data to identify specific cellular dysfunctions and to recommend specific interventions.

2. A system of claim 1:
Wherein CNRs are injected into a patient's arteries;
Wherein the CNRs identify arterial plaque depositions;
Wherein the CNRs map out the arterial system by creating detailed maps;
Wherein the CNRs deliver drugs to specific locations to reduce the arterial plaque depositions;
Wherein the CNRs abrasively remove arterial plaque deposits;
Wherein the CNRs continually report to an external computer with diagnostic feedback on their progress toward achieving their goal of reducing plaque; and
Wherein as a result of these interventions the patient's arterial plaque is reduced.

3. A system for managing automated collective nanorobots (CNRs), comprising:
A plurality of nanorobots, each nanorobot including program code configured to communicate and exchange information with other nanorobots;
Wherein the CNRs are self-organized to emulate a human pancreas;
Wherein the CNRs chemically process a patient's blood by modulating the blood sugar;
Wherein the CNRs divide into separate groups to emulate Alpha cells, Beta cells, Delta cells and polypeptide cells to create an artificial environment of the isles of Langerhans;
Wherein the CNRs use insulin, glucagon, somatastatin and polypeptides to emulate the paracrine feedback system to regulate blood sugar;
Wherein the CNRs conducts a glycation process of treating blood sugar;
Wherein nanorobots in the CNRs communicate with other nanorobots in the network to share information on the glycation process.

4. A system for managing automated collective nanorobots (CNRs), comprising:
A plurality of nanorobots, each nanorobot including program code configured to communicate and exchange information with other nanorobots;
Each nanorobot having an inner hull to carry a cargo of chemicals;
Each nanorobot having a doped outer hull to penetrate cellular membranes;
The nanorobots possessing mobility;
Wherein the CNR delivers nanocargoes to cells;
Wherein the CNR coordinates the network behavior of the collective to maximize the delivery of chemicals to specific cells using traveling salesman optimization algorithms;
Wherein the CNR is launched from a platform installed in a patient;
Wherein the CNR launches from the platform to deliver a chemical cargo and returns to the platform to receive a refill of chemicals; and
Wherein the CNR is installed in a virus to deliver a cargo to targeted cells.

5. A system of claim 4:
Wherein a CNR installed in the compartment of a stent is placed in a patient's arteries;
Wherein the CNR is activated to perform a function of delivering chemicals to cells;
Wherein the CNR is activated to remove arterial plaque in a specific sequence, with the highest priority blockage targeted initially and then the lower priority targets;
Wherein the CNR returns to the compartment in the stent when a mission is completed;
Wherein the CNR is time-released to perform different tasks;
Wherein the CNR is used to filter chemicals, cells, proteins and antigens from a position on the stent; and
Wherein the chemicals in the reservoir in the compartment in the stent are surgically replenished in endoscopic procedure.

6. A system of claim 4:
Wherein the CNRs identify the metastases of specific tumors;
Wherein the CNRs patrol specific tumors for metastases;
Wherein the CNRs identify the cells that receive the metastases from tumors;
Wherein the CNRs block the original tumors from spreading cancer cells to other tissues;
Wherein the CNRs identify and destroy the tumor cells that are spread to non-originating tissues;
Wherein the CNRs destroy the tumor cells by engulfing and rupturing them;
Wherein the CNRs transfer information about the mission to an external database for analysis; and
Wherein the CNRs target and tag the metastases to identify the metastases for immune system T cells.

7. A system of claim 4:
Wherein the CNRs access an implanted radioactive chemical supply;
Wherein the CNRs load the radioactive chemical supply into an inner cargo hold of the nanorobots;
Wherein the CNRs identify tumor cells;
Wherein the CNRs enter the tumor cells and disgorge the radioactive chemical inside the tumor cells;
Wherein the CNRs depart the tumor cells and return to obtain more radioactive chemicals; and
Wherein the tumor cells are destroyed.
